(19) United States
(12) Patent Application Publication
(10) Pub. No.: US 2012/0003159 A1
(43) Pub. Date: Jan. 5, 2012

(54) COMPOSITIONS AND METHODS FOR ENHANCING CONTRAST IN IMAGING

(75) Inventors: Ananth Annapragada, Marvel, TX (US); Russell M. Lebovitz, San Diego, CA (US); Ketankumar B. Ghaghada, Houston, TX (US)

(73) Assignees: Board of Regents of the University of Texas System, Austin, TX (US); Marval Biosciences, Inc, Houston, TX (US)

(21) Appl. No.: 13/256,672

(22) PCT Filed: Mar. 18, 2010
(86) PCT No.: PCT/US10/27795

§ 371 Date: Sep. 15, 2011

Related U.S. Application Data
(60) Provisional application No. 61/161,589, filed on Mar. 19, 2009.

(51) Int. Cl. A61K 49/04; B01J 13/20

(52) U.S. Cl. 424/9.4; 264/4.3

(57) ABSTRACT

Examples of compositions of liposomes and methods of making the same containing high concentrations of contrast-enhancing agents for computed tomography are provided. Example compositions of such liposomes, when administered to a subject, provide for increased contrast of extended duration, as measured by computed tomography, in the bloodstream and other tissues of the subject. Also provided are example methods for making the liposomes stable, that is, resistant to leakage of the contrast-enhancing agents, including the extrusion of the liposomes at high pressures and at high flow rates per total pore area of the extrusion filters.

### BACKGROUND

Some medical X-ray imaging techniques can detect variations in contrast of regions of interest in a subject, including different organs, tissues, cells and the like. To increase the contrast of regions of interest, some of the imaging techniques utilize the administration of one or more contrast-enhancing agents to a subject. The contrast-enhancing agents can accentuate existing differences in contrast between different areas of interest, or can produce differences in contrast where such differences do not exist without use of the agents.

There have been advancements in medical X-ray imaging, specifically relating to the instruments or machines used to detect the differences in contrast. These advancements include increases in the speed of the instruments, increases in the resolution of the instruments, and the like. These advancements have provided, in part, for new medical imaging methods. One example method, whole-body imaging, can yield information on the vasculature of the entire body of a subject.

Compared to advances in the instruments used for X-ray imaging, advances in contrast-enhancing agents have not been as forthcoming. Current contrast-enhancing agents for medical imaging using X-rays can have limitations for applications such as whole-body imaging due to, among other things, rapid clearance from the body of a subject, greater than desired extravasation, renal toxicity and inability to target specific areas of the body of a subject.

### BRIEF DESCRIPTION OF DRAWINGS

In the accompanying drawings, which are incorporated in and constitute a part of the specification, embodiments of contrast-enhancing agent formulations, pharmaceutical compositions containing the formulations, methods for making the formulations and methods for using the formulations in imaging are illustrated which, together with the detailed description given below, serve to describe the example embodiments of formulations, compositions, methods, and so on. It will be appreciated that the embodiments illustrated in the drawings are shown for the purpose of illustration and not for limitation. It will be appreciated that changes, modifications and deviations from the embodiments illustrated in the drawings can be made without departing from the spirit and scope of the invention, as disclosed below.

FIG. 1 illustrates an example method 100 of preparing liposomes containing or associated with contrast-enhancing agents.

FIG. 2 illustrates another example method 200 of preparing liposomes containing or associated with contrast-enhancing agents.

FIG. 3 illustrates another example method 300 of preparing liposomes containing or associated with contrast-enhancing agents.

FIG. 4 shows example results 400 from an in vitro stability test of embodiments of a liposomal iohexol formulation prepared using different extrusion processes, in saline at room temperature and 37° C.

FIG. 5 shows example results 500 from an in vitro stability test of embodiments of a liposomal iohexol formulation prepared using different extrusion processes and on different scales, in saline at room temperature and 37° C., and in bovine plasma.

FIG. 6 shows coronal maximum intensity projection images of in vivo tests of embodiments of a liposomal iohexol formulation prepared using different extrusion processes, demonstrating relative abdominal vascular enhancement and bladder enhancement.

### DETAILED DESCRIPTION

### Definitions

Definitions of selected terms or phrases are contained immediately following, and throughout the disclosure. The definitions include examples of various embodiments and/or forms of components that fall within the scope of a term and that may be used for implementation. The examples are not intended to be limiting and other embodiments may be implemented. Both singular and plural forms of all terms fall within each meaning.

“X-ray imaging,” as used herein, generally refers to any of a number of procedures using a source producing X-rays. Examples of X-ray imaging include computed tomography and the like.

“Computed tomography” or “CT” or “CAT,” as used herein, generally refers to procedures using a rotating X-ray instrument or machine to produce X-ray radiation and direct it through areas of a subject as the instrument rotates. The radiation that is not absorbed by the subject generally is detected and recorded as data. Generally, the data are sent to a computer which creates detailed cross-sectional images, or slices, of organs and body parts based on differential absorption of X-rays by different areas of the subject. CT of high resolution may be called “micro CT.”

“Whole body imaging,” as used herein, generally refers to methodologies for obtaining images, using CT for example, of the entire body of a subject. In one type of whole body imaging, the entire vasculature system may be examined Generally, imaging where the vasculature system is examined is called “blood pool imaging.”

### Description

This application describes example compositions comprising liposomes which contain or are associated with one or more contrast-enhancing agents. In one example, the liposomes contain or are associated with relatively high concentrations of contrast-enhancing agents. In one example, the liposomes contain one or more contrast-enhancing agents for X-ray imaging (e.g., CT imaging). In one example, the contrast-enhancing agents arc not radioactive.

In one example, the liposomes have one or more hydrophilic polymers attached to or associated with the liposomes. In one example, the hydrophilic polymers are attached to or associated with the surface of the liposomes. When administered to a subject, the liposomes can provide increased contrast in the body of a subject. In one example, the increased contrast lasts for an extended period of time.

This application also describes example pharmaceutical compositions that contain the liposomes and contrast-enhancing agents, and example methods of making the compositions of liposomes containing contrast-enhancing agents. The application also describes example methods of using the compositions in X-ray imaging.

### Contrast-Enhancing Agents

“Contrast-enhancing agent,” as used herein, generally refers to a substance that affects the attenuation, or the loss of intensity or power, of radiation as it passes through and interacts with a medium. It will be appreciated that contrast-enhancing agents may increase or decrease the attenuation. Generally, the contrast-enhancing agents referred to herein may increase the attenuation of radiation. In one example, the contrast-enhancing agents described herein are contrast-enhancing agents for X-ray imaging. In one example, the contrast-enhancing agents can be used for CT. In one example, the contrast-enhancing agents used herein are nonradioactive. In one embodiment, the contrast-enhancing agents can contain iodine and may be called “iodinated.”

Contrast-enhancing agents may be classified in various ways. In one classification, for example, iodinated contrast-enhancing agents can be water soluble (e.g., monoiodinated pyridine derivatives, di-iodinated pyridine derivatives, tri-iodinated benzene ring compounds, and the like), water-insoluble (e.g., propyliodone and the like) or oily (e.g., iodine in poppy seed oil, ethyl esters of iodinated fatty acids of poppy seed oil containing iodine, and the like).

In one example, a grouping of iodinated contrast-enhancing agents are water soluble. Present water soluble iodinated contrast-enhancing agents can be derivatives of tri-iodinated benzoic acid. These compounds can have one or more benzene rings. These compounds can be ionic or nonionic. Suitable, nonionic compounds include, but arc not limited to, metrizamide, iohexol, iopamidol, iopentol, iopromide, ioversol, iotrolan, iodixanol and others.

Suitable ionic compound contrast-enhancing agents may be weakly acidic (pKa of from approximately 4.0 to 6.5) or weakly basic (pKa of from approximately 6.5 to 8.5). Generally, acids are capable of giving up or donating one or more protons. In their protonated form, the acids are generally substantially electrically neutral or uncharged. In their unprotonated form, the acids are generally substantially negatively charged. Suitable weakly acidic agents can have one or more carboxyl groups. The carboxyl groups are capable of donating a proton. The carboxyl groups may be attached to a benzene ring and/or may be part of a benzoic acid. Examples of such benzoic acids include, but are not limited to, acetrizoate, diatrizoate, iodamide, ioglicate, iothalamate, ioxithalamate, metrizoate, sodium meglumine ioxaglate and others.

Generally, bases are capable of accepting one or more protons. In their protonated form, the bases are generally substantially positively charged. In their unprotonated form, the bases are generally substantially neutral or uncharged. Suitable weakly basic agents may have one or more primary amine groups. The amines are capable of accepting a proton. The weakly basic agents may be amides.

### Liposomes

“Liposomes,” as used herein, generally refer to spherical or roughly spherical particles containing an internal cavity. The walls of liposomes can include a bilayer of lipids. These lipids can be phospholipids. Numerous lipids and/or phospholipids may be used to make liposomes. One example are amphipathic lipids having hydrophobic and polar head group moieties which may form spontaneously into bilayer vesicles in water, as exemplified by phospholipids, or which may be stably incorporated into lipid bilayers, with their hydrophobic moiety in contact with the interior, hydrophobic region of the bilayer membrane, and their polar head group moiety oriented toward the exterior, polar surface of the membrane.

As used herein, “phospholipids” include, but arc not limited to, phosphatidic acid (PA), phosphatidylglycerol (PG), phosphatidylcholine (PC), egg phosphatidylcholine (EPC), lysophosphatidylcholine (LPC), phosphatidylethanolamine (PE), phosphatidylinositol (PI), phosphatidylserine (PS), and mixtures of two or more thereof The vesicle-forming lipids of this type may be lipids having two hydrocarbon chains, typically acyl chains, and a polar head group. Included in this class are phospholipids, such as phosphatidylcholine (PC), phosphatidylethanolamine (PE), phosphatidic acid (PA), phosphatidylglycerol (PG), phosphatidylinositol (PI), and sphingomyelin (SM), plus others. These phospholipids can be fully saturated or partially saturated. They can be naturally occurring or synthetic. In another example, lipids that can be included in the liposomes can be glycolipids.

The phospholipids used in the example liposomes described herein can be those where the two hydrocarbon chains are between about 14 and about 24 carbon atoms in length, and have varying degrees of unsaturation. Some examples of these phospholipids are given below. Although the phospholipids listed below may be used, alone or in combination with other phospholipids, the list is not intended to be complete. Other phospholipids not listed herein can also be used.

Phospholipids 1-Myristoyl-2-Palmitoyl-sn-Glycero-3-Phosphocholine, 1-Myristoyl-2-Stearoyl-sn-Glycero-3-Phosphocholine, 1-Myristoyl-2-Palmitoyl-sn-Glycero-3-Phosphocholine, 1-Myristoyl-2-Stearoyl-sn-Glycero-3-Phosphocholine, 1-Palmitoyl-2-Oleoyl-sn-Glycero-3-Phosphate (POPA), 1-Palmitoyl-2-Oleoyl-sn-Glycero-3-Phosphocholine, 1-Palmitoyl-2-Oleoyl-sn-Glycero-3-Phosphoethanolamine (POPE), 1-Palmitoyl-2-Oleoyl-sn-Glycero-3-[Phospho-rac-(1-glycerol)] (POPG), 1-Palmitoyl-2-Oleoyl-sn-Glycero-3-[Phospho-L-Serine] (POPS), 1-Palmitoyl-2-Linoleoyl-sn-Glycero-3-Phosphate, 1-Palmitoyl-2-Linoleoyl-sn-Glycero-3-Phosphocholine, 1-Palmitoyl-2-Linoleoyl-sn-Glycero-3-Phosphoethanolamine, 1-Palmitoyl-2-Linoleoyl-sn-Glycero-3-[Phospho-rac-(1-glycerol)], 1-Palmitoyl-2-Linoleoyl-sn-Glycero-3-[Phospho-L-Serine], 1-Palmitoyl-2-Arachidonoyl-sn-Glycero-3-Phosphate, 1-Palmitoyl-2-Arachidonoyl-sn-Glycero-3-Phosphocholine, 1-Palmitoyl-2-Arachidonoyl-sn-Glycero-3-Phosphoethanolamine, 1-Palmitoyl-2-Arachidonoyl-sn-Glycero-3-[Phospho-rac-(1-glycerol)], 1-Palmitoyl-2-Arachidonoyl-sn-Glycero-3-[Phosph-L-Serine], 1-Palmitoyl-2-Docosahexaenoyl-sn-Glycero-3-Phosphate, 1-Palmitoyl-2-Docosahexaenoyl-sn-Glycero-3-Phosphocholine, 1-Palmitoyl-2-Docosahexaenoyl-sn-Glycero-3-Phosphoethanolamine, 1-Palmitoyl-2-Docosahexaenoyl-sn-Glycero-3-[Phospho-rac-(1-glycerol-)], 1-Palmitoyl-2-Docosahexaenoyl-sn-Glycero-3-[Phospho-L-Serine], [0058]1-Stearoyl-2-Myristoyl-sn-Glycero-3-Phosphocholine, 1-Stearoyl-2-Palmitoyl-sn-Glycero-3-Phosphocholine, 1-Stearoyl-2-Oleoyl-sn-Glycero-3-Phosphate, 1-Stearoyl-2-Oleoyl-sn-Glycero-3-Phosphocholine, 1-Stearoyl-2-Oleoyl-sn-Glycero-3-Phosphoethanolamine, 1-Stearoyl-2-Oleoyl-sn-Glycero-3-[Phospho-rac-(1-glycerol], 1-Stearoyl-2-Oleoyl-sn-Glycero-3-[Phospho-L-Serine], 1-Stearoyl-2-Linoleoyl-sn-Glycero-3-Phosphate, 1-Stearoyl-2-Linoleoyl-sn-Glycero-3-Phosphocholine, 1-Stearoyl-2-Linoleoyl-sn-Glycero-3-Phosphoethanolamine, 1-Stearoyl-2-Linoleoyl-sn-Glycero-3-[Phospho-rac-(1-glycerol)], 1-Stearoyl-2-Linoleoyl-sn-Glycero-3-[Phospho-L-Serine], 1-Stearoyl-2-Arachidonoyl-sn-Glycero-3-Phosphate, 1-Stearoyl-2-Linoleoyl-sn-Glycero-3-Phosphocholine, 1-Stearoyl-2-Arachidonoyl-sn-Glycero-3-Phosphoethanolamine, 1-Stearoyl-2-Arachidonoyl-sn-Glycero-3-[Phospho-rac-(1-glycerol)], 1-Stearoyl-2-Arachidonoyl-sn-Glycero-3-[Phospho-L-Serine], -Stearoyl-2-Docosahexaenoyl-sn-Glycero-3-Phosphate, 1-Stearoyl-2-Docosahexaenoyl-sn-Glycero-3-Phosphocholine, 1-Stearoyl-2-Docosahexaenoyl-sn-Glycero-3-Phosphoethanolamine, 1-Stearoyl-2-Docosahexaenoyl-sn-Glycero-3-[Phospho-rac-(1-glycerol)-], 1-Stearoyl-2-Docosahexaenoyl-sn-Glycero-3-[Phospho-L-Serine], 1-Oleoyl-2-Myristoyl-sn-Glycero-3-Phosphocholine, 1-Oleoyl-2-Palmitoyl-sn-Glycero-3-Phosphocholine, 1-Oleoyl-2-Stearoyl-sn-Glycero-3-Phosphocholine, 1,2-Dimyristoyl-sn-Glycero-3-Phosphate (DMPA), 1,2-Dimyristoyl-sn-Glycero-3-Phosphocholine (DMPC), 1,2-Dimyristoyl-sn-Glycero-3-Phosphoethanolamine (DMPE), 1,2-Dimyristoyl-sn-Glycero-3-[Phospho-rac-(1-glycerol)] (DMPG), 1,2-Dimyristoyl-sn-Glycero-3-[Phospho-L-Serine] (DMPS), 1,2-Dipentadecanoyl-sn-Glycero-3-Phosphocholine, 1,2-Dipalmitoyl-sn-Glycero-3-Phosphate (DPPA), 1,2-Dipalmitoyl-sn-Glycero-3-Phosphocholine (DPPC) , 1,2-Dipalmitoyl-sn-Glycero-3-Phosphoethanolamine (DPPE), 1,2-Dipalmitoyl-sn-Glycero-3-[Phospho-rac-(1-glycerol)] (DPPG), 1,2-Dipalmitoyl-sn-Glycero-3-[Phospho-L-Serine) (DPPS), 1,2-Diphytanoyl-sn-Glycero-3-Phosphate, 1,2-Diphytanoyl-sn-Glycero-3-Phosphocholine, 1,2-Diphytanoyl-sn-Glycero-3-Phosphoethanolamine, 1,2-Diphytanoyl-sn-Glycero-3-[Phospho-rac-(1-glycerol)], 1,2-Diphytanoyl-sn-Glycero-3-[Phospho-L-Serine], 1,2-Diheptadecanoyl-sn-Glycero-3-Phosphocholine, 1,2-Distearoyl-sn-Glycero-3-Phosphate (DSPA), 1,2-Distearoyl-sn-Glycero-3-Phosphocholine (DSPC), 1,2-Distearoyl-sn-Glycero-3-Phosphoethanolamine (DSPE), 1,2-Distearoyl-sn-Glycero-3-[Phospho-rac-(1-glycerol)] (DSPG), 1,2-Distearoyl-sn-Glycero-3-[Phospho-L-Serine], 1,2-Dibromostearoyl-sn-Glycero-3-Phosphocholine, 1,2-Dinonadecanoyl-sn-Glycero-3-Phosphocholine, 1,2-Diarachidoyl-sn-Glycero-3-Phosphocholine, 1,2-Diheneicosanoyl-sn-Glycero-3-Phosphocholine, 1,2-Dibehenoyl-sn-Glycero-3-Phosphocholine, 1,2-Ditricosanoyl-sn-Glycero-3-Phosphocholine, 1,2-Dilignoceroyl-sn-Glycero-3-Phosphocholine, 1,2-Dimyristoleoyl-sn-Glycero-3-Phosphocholine, 1,2-Dimyristelaidoyl-sn-Glycero-3-Phosphocholine, 1,2-Dipalmitoleoyl-sn-Glycero-3-Phosphocholine, 1,2-Dipalmitelaidoyl-sn-Glycero-3-Phosphocholine, 1,2-Dipalmitoleoyl-sn-Glycero-3-Phosphoethanolamine, 1,2-Dioleoyl-sn-Glycero-3-Phosphocholine (DOPC), 1,2-Dioleoyl-sn-Glycero-3-Phosphate (DOPA), 1,2-Dioleoyl-sn-Glycero-3-Phosphocholine (DOPC), 1,2-Dioleoyl-sn-Glycero-3-Phosphoethanolamine (DOPE), 1,2-Dioleoyl-sn-Glycero-3-[Phospho-rac-(1-glycerol)] (DOPG), 1,2-Dioleoyl-sn-Glycero-3-[Phospho-L-Serine] (DOPS), 1,2-Dielaidoyl-sn-Glycero-3-Phosphocholine, 1,2-Dielaidoyl-sn-Glycero-3-Phosphoethanolamine, 1,2-Dielaidoyl-sn-Glycero-3-[Phospho-rac-(1-glycerol)], 1,2-Dilinoleoyl-sn-Glycero-3-Phosphate, 1,2-Dilinoleoyl-sn-Glycero-3-Phosphocholine, 1,2-Dilinoleoyl-sn-Glycero-3-Phosphoethanolamine, 1,2-Dilinoleoyl-sn-Glycero-3-[Phospho-rac-(1-glycerol)], 1,2-Dilinoleoyl-sn-Glycero-3-[Phospho-L-Serine], 1,2-Dilinolenoyl-sn-Glycero-3-Phosphocholine, 1,2-Dilinolenoyl-sn-Glycero-3-Phosphoethanolamine, 1,2-Dilinolenoyl-sn-Glycero-3-[Phospho-rac-(1-glycerol)], 1,2-Dieicosenoyl-sn-Glycero-3-Phosphochone, 1,2-Diarachidonoyl-sn-Glycero-3-Phosphate, 1,2-Diarachidonoyl-sn-Glycero-3-Phosphocholine, 1,2-Diarachidonoyl-sn-Glycero-3-Phosphoethanolamine, 1,2-Diarachidonoyl-sn-Glycero-3-[Phospho-rac-(1-glycerol)], 1,2-Diarachidonoyl-sn-Glycero-3-[Phospho-L-Serine], 1,2-Dierucoyl-sn-Glycero-3-Phosphocholine, 1,2-Didocosahexaenoyl-sn-Glycero-3-Phosphate, 1,2-Didocosahexaenoyl-sn-Glycero-3-Phosphocholine, 1,2-Didocosahexaenoyl-sn-Glycero-3-Phosphoethanolamine, 1,2-Docosahexaenoyl-sn-Glycero-3-[Phospho-rac-(1-glycerol)], 1,2-Didocosahexaenoyl-sn-Glycero-3-[Phospho-L-Serine], and 1,2-Dinervonoyl-sn-Glycero-3-Phosphocholine.

The liposome composition can be formulated to include amounts of fatty alcohols, fatty acids, and/or cholesterol esters or other pharmaceutically acceptable excipients. For example, the liposomes can include lipids that can stabilize a vesicle or liposome composed predominantly of phospholipids. For example, cholesterol between about 25 to 40 mole percent may be used.

In one embodiment, the type of liposomes used may be “sterically stabilized liposomes.” Sterically stabilized liposomes can include a surface that contains or is coated with flexible water soluble (hydrophilic) polymer chains. These polymer chains may prevent interaction between the liposomes and blood plasma components, the plasma components playing a role in uptake of liposomes by cells of the blood and removal of the liposomes from the blood. Sterically stabilized liposomes may avoid uptake by the organs of the mononuclear phagocyte system, primarily the liver and spleen (reticulendothelial system or RES). Such sterically stabilized liposomes may also be called “long circulating liposomes.”

Sterically stabilized liposomes can contain lipids or phospholipids that are derivatized with a polymer chain. The lipids or phospholipids that may be used generally may be any of those described above. One exemplary phospholipid is phosphatidylethanolamine (PE) with a reactive amino group which may be convenient for coupling to the activated polymers. An exemplary PE may be distearyl PE (DSPE).

Examples of polymers that are suitable for use in sterically stabilized liposomes include, but are not limited to, the hydrophilic polymers polyvinylpyrrolidone, polymethyloxazoline, polyethyloxazoline, polyhydroxypropyl methacrylamide, polymethacrylamide, polydimethylacrylamide, polylactic acid, polyglycolic acid, and derivatized celluloses, like hydroxymethylcellulose or hydroxyethylcellulose. Polylysine may be used. Lipid-polymer conjugates containing these polymers attached to a suitable lipid, such as PE, may be used. Other example polymers can be used.

In one embodiment, the polymer in the derivatized lipid or phospholipid can be polyethylene glycol (PEG). The PEG can have any of a variety of molecular weights. In one example, the PEG chain may have a molecular weight between about 1,000-10,000 daltons. Once a liposome is formed, the PEG chains may provide a surface coating of hydrophilic chains sufficient to extend the blood circulation time of the liposomes in the absence of such a coating. Such liposomes may be called “PEGylated liposomes.” PEGylated liposomes can include so-called STEALTH® liposomes, provided by ALZA Corporation.

PEGylated liposomes may also include liposomes with PEG on their surface, where the PEG may be released from the liposome at some time after administration of the liposomes to a subject. In one example, there can be one or more bonds or linkages attaching the PEG, or other hydrophilic polymer, to the liposome surface and/or lipid molecules comprising the liposome surface. In one example, the bonds or linkages can be cleaved, providing for separation of the PEG from the liposome. For example, PEG may be attached to a lipid by one or more disulfide bonds. The disulfide bonds may be cleaved by free thiol, releasing the PEG from the liposome. Other types of cleavable links or bonds can be used to attach the polymers to the liposomes. Other types of agents or compounds can be used to cleave the bonds or linkages.

In one example, the liposomes used can have a composition of between about 60 and 75 mole % of one or more of the phospholipids with carbon chains between about 14-24 in length, as described above. A fraction of these phospholipids may be attached to one or more hydrophilic polymers such that between about 1 and 20 mole % of the liposome composition is phospholipid derivatized with polymer chains. In addition, the liposomes used may have between about 25 and 40 mole % cholesterol, or fatty alcohols, fatty acids, and/or other cholesterol esters or other pharmaceutically acceptable excipients, generally for the purpose of stabilizing the liposomes.

In another example, the liposomes can have a molecule or molecules, commonly called a “ligand,” which may be accessible from the surface of the liposome, that may specifically bind or attach to, for example, one or more molecules or antigens. These ligands may direct or target the liposomes to a specific cell or tissue and may bind to a molecule or antigen on or associated with the cell or tissue. The ligand may be an antibody or antibody fragment. The antibody may be a monoclonal antibody or fragment. Such liposomes may be of a type called “targeted liposomes.”

In one example, targeted liposomes can have lipids or phospholipids which have been modified for coupling antibody molecules to the liposome outer surface. These modified lipids may be of different types. The modified lipid may contain a spacer chain attached to the lipid. The spacer chain may be a hydrophilic polymer. The hydrophilic polymer may typically be end-functionalized for coupling antibody to its functionalized end. The functionalized end group may be a maleimide group, for selective coupling to antibody sulfhydryl groups. Other functionalized end groups may include bromoacetamide and disulfide groups for reaction with antibody sulfhydryl groups, activated ester and aldehyde groups for reaction with antibody amine groups. Hydrazide groups are reactive toward aldehydes, which may be generated on numerous biologically relevant compounds. Hydrazides may also be acylated by active esters or carbodiimide-activated carboxyl groups. Acyl azide groups reactive as acylating species may be easily obtained from hydrazides and permit the attachment of amino containing ligands.

In another example, the phospholipid can be modified by a biotin molecule. To attach the antibody molecule to the biotinylated liposome surface, once the liposome is formed, the antibody molecule may also be modified with biotin and then incubated in the presence of the avidin. Biotinylated lipids, such as biotinylated PE, may be commercially available.

In another example, lipids can be modified by a substrate for use in binding a targeting molecule to a liposome surface. Typically, substrates, as exemplified with biotin, may be relatively small, less than about 5,000 daltons for example, to allow their incorporation into multilamellar liposomes with a minimum of disruption of the lipid bilayer structures. The substrate may be one capable of binding irreversibly to a targeting molecule, to ensure that the targeting molecule remains bound to the liposomes over its lifetime in the bloodstream.

### Preparation of Liposomes Containing Contrast-Enhancing Agents

Liposomes can be prepared by a variety of methods. Example methods include, but are not limited to, hydration of dried lipids, introduction of a volatile organic solution of lipids into an aqueous solution causing evaporation of the organic solution, and dialysis of an aqueous solution of lipids and detergents or surfactants to remove the detergents or surfactants, and other methods.

Liposomes can contain or may be associated with one or more contrast-enhancing agents. In one example, the liposomes may contain the contrast-enhancing agents. In the process of making liposomes, the contrast-enhancing agents may be added at any desired time. For example, contrast-enhancing agents may be associated with components of liposomes before liposomes are formed. Contrast-enhancing agents may be combined with liposome components at the time the liposomes are made. Contrast-enhancing agents may also be added after the liposomes are formed. Other methods of associating contrast-enhancing agents with liposomes may exist. Generally, contrast-enhancing agents which are hydrophilic in nature may be located or associated with the internal cavity of the liposome particles. Contrast-enhancing agents which are lipophilic in nature may be located or associated with the lipid bilayer of liposome particles. Generally, the contrast-enhancing agents herein are located or associated with the internal cavity of the liposome. The example liposomes contain at least 30 mg iodine/milliliter (I/mL) of liposome suspension when iodinated contrast enhancing agents are used. One example of the liposomes can contain between about 35 and about 250 mg I/mL of liposome suspension. One example of the liposomes can contain between about 37 and about 200 mg I/mL of liposome suspension. One example of the liposomes can contain between about 80 and about 160 mg I/mL of liposome suspension. One example of the liposomes can contain between about 100 and about 120 mg I/mL of liposome suspension. One example of the liposomes can contain between about 85 and about 100 mg I/mL of liposome suspension. One example of the liposomes can contain more than about 100 mg I/mL of liposome suspension.

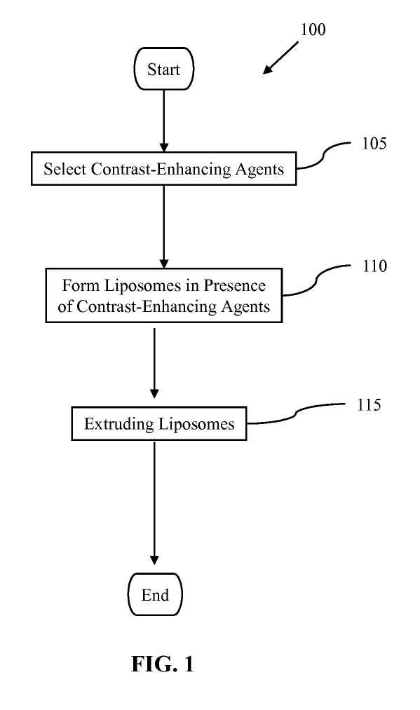

There are a variety of methods for loading the contrast-enhancing agents into the liposomes. Example methods may be better appreciated with reference to the flow diagrams of FIGS. 1-3. While for purposes of simplicity of explanation, the illustrated methodologies are shown and described as a series of blocks, it is to be appreciated that the methodologies are not limited by the order of the blocks, as some blocks may occur in different orders and/or concurrently with other blocks from that shown and described. Moreover, less than all the illustrated blocks may be required to implement an example methodology. Blocks may be combined or separated into multiple components. Furthermore, additional and/or alternative methodologies can employ additional, not illustrated blocks. While the figures illustrate various actions occurring in serial, it is to be appreciated that various actions could occur concurrently, substantially in parallel, and/or at substantially different points in time. The diagrams of FIGS. 1-3 are not intended to limit the implementation of the described examples.

Illustrated in FIG. 1 is an example method 100 for preparing liposomes containing or associated with contrast-enhancing agents. The method may include selecting one or more contrast-enhancing agents to be used (block 105). The method may also include forming liposomes in the presence of the one or more contrast-enhancing agents (block 110). Generally, the step illustrated as block 110 may be performed using the methods described earlier for preparing liposomes. These methods may include hydration of dried lipids, introduction of a volatile organic solution of lipids into an aqueous solution causing evaporation of the organic solution, dialysis of an aqueous solution of lipids and detergents or surfactants to remove the detergents or surfactants, and others.

Remarkably, the liposomes may be significantly stabilized by extruding the liposomes at high pressure, at a high flow rate, or both (block 115). The extrusion pressure and/or the flow rate may be manipulated in several ways. Two example ways are changing the pore size of the filter and changing the configuration of the filter used. Generally, the flow rates are calculated by measuring the flow rate per total pore area (LPH/m²). The pore size may define the pore density of the filters used, as opposed to the overall size of the filter. It has been found that the higher the flow rate per pore area and the higher the pressure at which the solution is extruded, the more stable the resulting liposomes will be.

In one embodiment, a “high” flow rate may include a flow rate per total pore area (LPH/m²) of at least about 800-1,000 LPH/m². In another embodiment, a “high” flow rate may include a flow rate per total pore area of from about 1,000-5,000 LPH/m². In another embodiment, a “high” flow rate may include a flow rate per total pore area of from about 5,000-15,000 LPH/m². In another embodiment, a “high” flow rate may include a flow rate per total pore area of from about 15,000-25,000 LPH/m². In another embodiment, a “high” flow rate may include a flow rate per total pore area of from about 25,000-50,000 LPH/m². In yet another embodiment, a “high” flow rate may include a flow rate per total pore area of greater than 50,000 LPH/m².

In one embodiment, a “high” extrusion pressure may include a pressure of at least 40 psi. In another embodiment, a “high” extrusion pressure may include a pressure of at least about 50 psi. In another embodiment, a “high” extrusion pressure may include a pressure of from about 50-100 psi. In another embodiment, a “high” extrusion pressure may include a pressure of greater than about 100 psi. In another embodiment, a “high” extrusion pressure may include a pressure of from about 100-150 psi. In another embodiment, a “high” extrusion pressure may include a pressure of from about 100-200 psi. In another embodiment, a “high” extrusion pressure may include a pressure of greater than about 200 psi. In another embodiment, a “high” extrusion pressure may include a pressure of from about 200-250 psi. In another embodiment, a “high” extrusion pressure may include a pressure of about 240 psi. In another embodiment, a “high” extrusion pressure may include a pressure of greater than 250 psi.

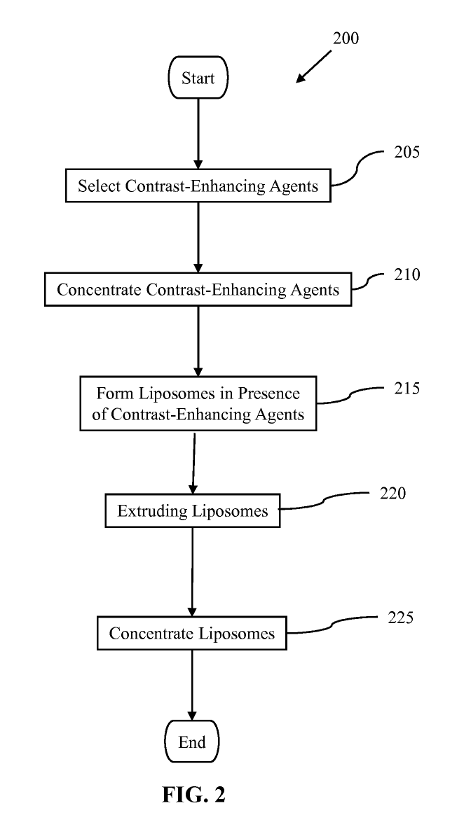

Illustrated in FIG. 2 is another example method 200 for preparing liposomes containing or associated with contrast-enhancing agents. The method may include selecting one or more contrast-enhancing agents to be used (block 205). The method may also include concentrating the one or more contrast-enhancing agents (block 210). The method may also include forming liposomes in the presence of the one or more contrast-enhancing agents (block 215). The method may also include extruding the liposomes, as described above, (block 220) and concentrating the liposomes (block 225).

Concentrating the one or more contrast-enhancing agents (block 210) can be performed using a variety of methods. In one example, a commercially available solution of one or more contrast-enhancing agents may be concentrated using the methods. In one example, the contrast-enhancing agents may be precipitated from a solution and the precipitated contrast-enhancing agents suspended in a liquid at a concentration higher than in the original solution. In another example, the contrast-enhancing agents in a solution may be concentrated by evaporation. One example of evaporation may be rotary evaporation. Other methods may be used. In one example, a solution of contrast-enhancing agents may be concentrated by at least 10%. In one example, a solution of contrast enhancing agents may be concentrated by 100% (i.e., 2-fold) or more. In another example, solid forms of the contrast enhancing agents may be dissolved in a liquid at a relatively high concentration (e.g., at a higher concentration than in commercially available solutions). In one example, heating may be used to increase the solubility of the contrast-enhancing agents in the solution. In another example, a solvent may be used in which the contrast-enhancing agents may be more soluble than in another solvent.

It will be appreciated that the viscosity of a liposome suspension generally is determined by the concentration of liposomes and generally is not determined by the viscosity of the liposome contents. For example, contrast-enhancing agents that have been encapsulated into liposomes may form a gel phase or even crystallize inside the liposomes (e.g., if the temperature is lowered). Generally, this may not affect the liposome suspension and may facilitate the stability of the liposome suspension (e.g., by reducing the probability of leakage of the contrast-enhancing agents from the liposomes).

After the liposomes are made and arc in solution, the solution of liposomes may be concentrated to obtain a more concentrated solution of liposomes by decreasing the volume of the solution without substantially changing the number of liposomes in the solution. Concentrating the liposomes (block 225) can be performed using a variety of methods. When the liposomes are in an aqueous solution, concentration by removal of water may be called dewatering. One example method of dewatering can be diafiltration. In one example of diafiltration, a suspension of liposomes in a liquid may be passed through a filter or membrane to decrease the amount of liquid in which an amount of liposomes is suspended. Diafiltration may also be used to remove unencapsulated iodine from the suspension of liposomes.

Other example methods can include ion exchange, washing of the liposomes using ultracentrifugation, dialysis, and so on. These methods can result in example liposome suspensions with concentrations of between about 35-250 mg I/mL of liposome suspension. One example of the liposomes can contain between 37 and 200 mg I/mL of liposome suspension. One example of the liposomes can contain more than 100 mg I/mL of liposome suspension. These methods may also remove impurities from a suspension of liposomes. In one example, the impurities may include contrast-enhancing agents that have not been encapsulated into or associated with liposomes.

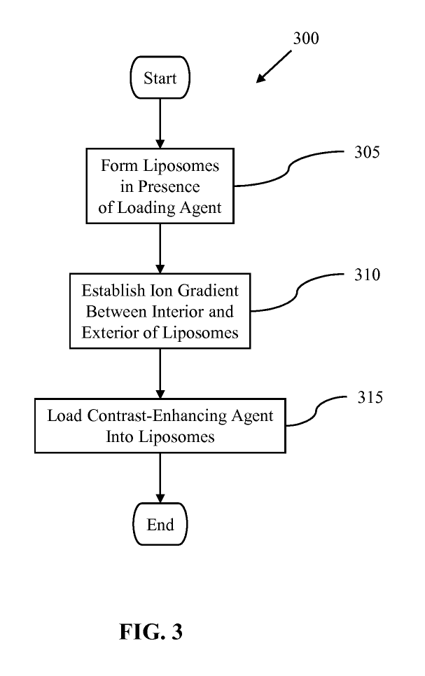

Illustrated in FIG. 3 is another example method 300 for preparing liposomes containing or associated with contrast-enhancing agents. The method 300 may include forming liposomes in the presence of a loading agent (block 305). The method may also include establishing an ion gradient between the interface and exterior of the liposomes (block 310). The method may also include loading one or more ionic iodinated benzenes into the liposomes (block 315).

The method illustrated in FIG. 3 may be of a type or class referred to as active or remote loading methods. In one example of active or remote loading, the contrast-enhancing agent or agents to be contained by or within the liposomes (e.g., contrast-enhancing agents) may enter liposomes after the liposomes have been formed or partially formed. Such formed liposomes generally are those whose process of making is completed. Partially formed liposomes may not have completed the making process.

In one example method, an ion gradient can be established from or between the outside of the liposome and the inside of the liposome (e.g., the concentration of one or more ions outside the liposomes is different than the concentration inside the liposomes) of the formed liposomes. The contrast-enhancing agent to be loaded into the liposomes can move from the outside of the liposomes to the inside of the liposomes. This movement may be due to movement of the contrast-enhancing agent through the membranes of the liposomes. Generally, contrast-enhancing agents capable of moving through membranes may be substantially neutral in electrical charge or uncharged. This movement may be based on a concentration gradient (e.g., a greater concentration of the contrast-enhancing agent outside the liposomes than inside the liposomes). This movement may be based on an ion gradient. This movement may be based on other factors or combinations of various factors. Once inside the liposomes, the different ion concentration inside the liposomes as compared to outside the liposomes may retard or prevent the contrast-enhancing agent from moving out of the liposomes. In one example, the different ion concentration inside the liposomes as compared to outside the liposomes can chemically alter the contrast-enhancing agent such that its movement out of the liposomes is retarded or prevented.

One example ion gradient can be a pH gradient. Hydrated liposomes may have a selected internal and external pH. This pH may have been selected based on the pH of the environment in which the liposomes were formed. The external solution in which the hydrated liposomes are present may then be titrated until a selected pH different from the internal pH is obtained. The external solution may also be exchanged with another solution of a selected pH different from the internal pH. For example, the original external solution in which the liposomes are present may have a pH of 5.5 and then be titrated or exchanged for a solution that may have a pH of 8.5. Once a contrast-enhancing agent enters into the liposomes, a contrast-enhancing agent inside the liposome may be chemically altered by accepting or donating one or more protons. A contrast-enhancing agent that has accepted or donated one or more protons may be charged. The charged contrast-enhancing agents may be unable or inhibited in their ability to pass through the liposome membrane. In these liposomes, the contrast-enhancing agents may be unable to exit or have a reduced ability to exit the liposomes.

In another example of active or remote loading, the formed or partially formed liposomes may contain a loading agent. For example, the liposomes may be formed in the presence of the loading agent. The loading agent may assist or facilitate entry of contrast-enhancing agents into the liposomes. The loading agent may facilitate establishing a certain condition inside the liposomes, such as a concentration of hydrogen ions for example. The loading agent may facilitate chemical alteration of a contrast-enhancing agent, such as facilitating the contrast-enhancing agent accepting or donating one or more protons. The loading agent may prevent or retard contrast-enhancing agents that enter the liposomes from leaving the liposomes.

In one example approach, a weakly acidic contrast-enhancing agent (pKa of from approximately 4.0 to 6.5) is loaded into liposomes. Such an agent may be weakly amphiphatic. The weakly acidic agent may be substantially uncharged in its protonated form. The weakly acidic agent may be substantially negatively charged in its unprotonated form. Generally, such weakly acidic agents may have one or more free carboxyl groups. Such free carboxyl groups may be ionizable in that they may donate a proton. Example weakly acidic contrast-enhancing agents may include acetrizoate, diatrizoate, iodamide, ioglicate, iothalamate, ioxithalamate, metrizoate, ioxaglate, and others

In one example of this approach, liposomes can be formed in the presence of calcium acetate (e.g., (CH₃COO)₂Ca). The calcium acetate may be a loading agent. Calcium acetate is present inside the liposomes and in the external solution. The calcium acetate may then be removed from the phase exterior to the liposomes, by dilution for example. Calcium acetate inside the liposomes may dissociate into calcium ion and acetate ions. The acetate ions may combine with water inside the liposomes to yield acetic acid and hydroxide ion. Dilution of the solution external to the liposomes may cause acetic acid inside the liposomes to diffuse out of the liposomes, into the external solution, leaving hydroxide ions inside the liposomes. This may create a pH gradient in which the interior of the liposomes are more basic than the exterior of the liposomes. Addition of a weakly acidic contrast-enhancing agent to an exterior phase at a pH where a significant amount of the weakly acidic contrast-enhancing agent is protonated and uncharged may result in the contrast-enhancing agent moving into the interior of the liposomes. Such movement may be due to an outside-to-inside concentration gradient of the agent. Such movement may be due to forces favoring osmolar equilibrium as ammonia moves out of the liposomes. Such movement may be due to other or additional forces or combinations of such forces. When the contrast-enhancing agent moves to the interior of the liposomes, the contrast-enhancing agent may donate one or more protons, becoming negatively charged, and may be retarded or prevented from moving out of the liposome. Additions to, substitutions and variations of this approach may exist.

In one example approach, a weakly basic contrast-enhancing agent (pKa of from approximately 6.5 to 8.5) agent is loaded into liposomes. Such an agent may be weakly amphiphatic. The weakly basic agent generally is uncharged at or around neutral pH. The weakly basic agent may be substantially uncharged in its unprotonated form. The weakly basic agent may be substantially positively charged in its protonated form. Generally, such weakly basic agents may have one or more primary amine groups. Such primary amine groups may be ionizable in that they may accept a proton. Such weakly basic agents may be amides.

In one example of this approach, liposomes can be formed in the presence of ammonium sulfate ((NR₄)SO₄). The ammonium sulfate may be a loading agent. Ammonium sulfate is present inside the liposomes and in the external solution. The ammonium sulfate may then be removed from the phase exterior to the liposomes, by dilution for example Ammonium sulfate inside the liposomes may dissociate into ammonium ions (NH₄⁺) and sulfate ions (SO₄⁻). Ammonium ions inside the liposomes may dissociate into ammonia and hydrogen ions. Dilution of the solution external to the liposomes may cause ammonia inside the liposomes to diffuse out of the liposomes, into the external solution, leaving hydrogen ions inside the liposomes. This may create a pH gradient in which the interior of the liposomes are more acidic than the exterior of the liposomes. Addition of a weakly basic contrast-enhancing agent to an exterior phase at a pH where a significant amount of the weakly basic contrast-enhancing agent is unprotonated and uncharged may result in the contrast-enhancing agent moving into the interior of the liposomes. Such movement may be due to an outside-to-inside concentration gradient of the contrast-enhancing agent. Such movement may be due to forces favoring osmolar equilibrium as ammonia moves out of the liposomes. Such movement may be due to other or additional forces or combinations of such forces. When the contrast-enhancing agent moves to the interior of the liposomes, the contrast-enhancing agent may accept one or more protons, becoming positively charged, and may be retarded or prevented from moving out of the liposome. Additions to, substitutions and variations of this approach may exist. A variety of other active or remote loading methods may also exist.

After liposomes arc made, techniques for manipulating the liposomes can be used. For example, a preparation of liposomes made by standard techniques may vary in size and lamellarity (i.e., wall thickness) after it is made. Techniques like subjecting the liposomes to a high shearing force, extrusion of the liposomes through membranes, or sonication of the liposomes may be used either to select liposomes of a desired size or modify the liposomes so that they have a desired size. After manipulation of liposomes by these methods, the size distribution of the liposomes may be measured to ensure that liposomes of the desired size have been obtained. Techniques like as Fraunhofer diffraction and dynamic light scattering (DLS) may be used to measure the size distribution of the liposomes. These techniques generally measure an equivalent spherical diameter which, in the case of Fraunhofer diffraction, may be the diameter of a sphere with the same light scattering properties as the measured liposomes. In the case of DLS, equivalent spherical diameter may be the diameter of a sphere with the same diffusion coefficient as the measured liposomes. Generally, the example liposomes have an average diameter of 150 nm or less. Example preparations of liposomes may have an average diameter of approximately 120 nm or less. Example preparations of liposomes may have an average diameter of approximately 100 nm or less. It will be appreciated that other sizes can be used.

In one embodiment, a nano-scale liposomal formulation carrying over 30 mg of iohexol per mL of liposome is formulated using passive loading. In this formulation, the lipid composition of the bilayer is adjusted as described below to allow this amount of contrast-enhancing agent to be encapsulated. In one example, using pure DPPC (1,2-dipalmitoyl-sn-glycero-3-phosphatidylcholine) of C16 chain length, with about 40 mole % cholesterol and 5 mole % mPEG-DSPE (N-(carbonylmethoxypolyethyleneglycol 2000)-1,2-distearoyl-sn-glycero-3-phosphatidylethanolamine) (the polyethylene glycol-conjugated lipid that confers long circulating properties), the encapsulation of active molecules inside the liposomes is increased by 20% over what is possible using hydrogenated Soy PC(HSPC), a mixture of C16 and C18 lipids, or pure DSPC (1,2-distearoyl-sn-glycero-3-phosphocholine) of C18 chain length. Using a formulation of 55 mole % DPPC, 40 mole % cholesterol and 5 mole % mPEG-DSPE and an iohexol solution of 350 mg I/mL, an overall concentration of over 30 mg I/mL is achieved, with an average liposomal diameter of 100.6.+−0.3 nm, as determined by DLS.

In another embodiment, a liposomal formulation carrying over 80 mg of iohexol per mL of liposome is formulated using passive loading. In this formulation, an iohexol solution of 350 mg I/mL is concentrated to at least 400-450 mg I/mL and used to prepare liposomes as described in the previous paragraph. After the liposomes are obtained, the suspension of the liposomes is concentrated. Using this formulation, liposome suspensions with a concentration of over 85 mg I/mL are obtained.

### Pharmaceutical Compositions and Administration to Subjects

The liposomes containing and/or associated with one or more contrast-enhancing agents can be part of a pharmaceutical composition suitable for administration to a subject. The compositions generally are administered using a route that delivers the composition to an area of interest. In one example, the compositions of contrast-enhancing agents are administered parenterally to the subject, such as through intravenous, intraarterial, subcutaneous, or other route of injection.

The formulation of the particular pharmaceutical composition generally will depend on the method by which the composition is administered to a patient. It will be appreciated that the pharmaceutical compositions can include salt, buffering agents, preservatives, other vehicles and, optionally, other agents. Compositions suitable for parenteral administration may comprise a sterile, pyrogen-free, aqueous or oleaginous preparation which is generally isotonic with the blood of the subject. This aqueous preparation may be formulated according to known methods using suitable dispersing or wetting agents and suspending agents. The sterile injectable preparation also may be a sterile injectable solution or suspension in a non-toxic parenterally-acceptable diluent or solvent. Among acceptable vehicles and solvents that may be employed are water, Ringer's solution, and isotonic sodium chloride or other salt, dextrose, phosphate buffered saline and the like, or combinations thereof

The pharmaceutical compositions used may also contain stabilizers, preservatives, buffers, antioxidants, or other additives. In addition, sterile, fixed oils may be employed as a solvent or suspending medium. In addition, fatty acids such as oleic acid may be used in the preparation of injectables. Carrier formulations suitable for the administrations may be found in Remington's Pharmaceutical Sciences, Mack Publishing Co., Easton, Pa. The pharmaceutical compositions may conveniently be presented in unit dosage form.

Parenteral administration contemplates the use of a syringe, catheter or similar device, which delivers the pharmaceutical composition to a site. Delivery may result, at least initially, in the pharmaceutical composition being systemically distributed throughout the circulatory system of the subject.

Generally, the pharmaceutical compositions are administered to the subject at a point in time before the imaging of the subject is performed, although the compositions may also be administered during the imaging. The amount of the pharmaceutical compositions administered preferably results in increased contrast of one or more tissues of the subject. Ultimately, the attending physician or technician generally will decide the amount of pharmaceutical composition to administer to the subject. Generally, the increase in contrast can be any level above what is present without use of the contrast-enhancing agents in the pharmaceutical compositions. Example increases in contrast of at least about 50 HU, at least about 100 HU or more, to one or more organ systems, including the vasculature, may be obtained.

### Applications

The compositions of liposomes containing contrast-enhancing agents or pharmaceutical compositions thereof, when administered to a subject, can maintain a level of contrast-enhancing agent in the blood and/or organs of a subject that results in an increased contrast and is detectable by X-ray imaging techniques. The increase in contrast may be detectable for an extended period of time. Depending on the particular application, the compositions described herein may have half lives in the circulation of from minutes to hours, to even days. In one example, half lives in the circulation of from 8 to 24 hours may be obtained. In one example, an administered composition provides an enhanced contrast that may remain detectable at least 30 minutes after administration. In another example, an administered composition provides an enhanced contrast that may remain detectable at least 5 minutes after administration. Many applications, including those in anatomic, functional and molecular imaging may be possible. For example, use of the compositions described herein may have applications in cardiology, oncology, neurology and other areas.

In one embodiment, blood pool imaging can be used to detect and, in some cases, quantify ischemia. For example, because injection of the pharmaceutical compositions generally alters the contrast of the entire vasculature, reduced blood flow as is present in ischemia may be detected. A variety of types of ischemia may be detected, including that causing ischemic bowel disease, pulmonary embolism, and types of ischemia that produce cardiomyopathy, and others. In other applications, aneurysms may also be detected.

In one embodiment, the compositions described herein can be used in cardiac imaging to detect, examine and/or assess stenosis, and the therapy or remediation of stenosis, as occurs in angioplasty, for example. The utility of such techniques may be enhanced through the use of contrast-enhancing agent preparations, such as those described herein.

In one embodiment, the compositions described herein can be used to detect myocardial microcirculatory insufficiencies. Myocardial microcirculation is known to display signs of obstruction before the epicardial arteries show signs of obstruction. Therefore, detection of obstruction in the myocardial microcirculation may be an earlier detector of atherosclerosis in presymptomatic, at-risk patients, than conventional methods. The compositions described herein may facilitate detection of obstructions in the myocardial microcirculation.

In another embodiment, the compositions described herein can be used to detect and characterize a wide range of tumors and cancers. These applications may be facilitated by the property of sterically stabilized liposomes being present for extended periods of time in the circulation and to extravasate at regions where the vasculature is “leaky,” such as in tumors, for example. The leakiness of the vasculature in tumors may be attributed to the high proportion of neovasculature, the result of continuing angiogenesis as the tumor grows in size. Upon encountering such leaky vasculature, liposomes may leave the circulation, driven with the extravasate fluid, by hydrostatic pressure. Such liposomes generally do not return to the circulation after extravasation since the pressure gradient opposes such return. Such methods may be used to detect both primary and metastatic tumors.

In other embodiments, the compositions can be used for “staging” and/or classification of tumors. These applications may depend on, among other things, differences in the “leakiness” of the vasculature of a given tumor or cancer at different stages of progression.

In one embodiment, the compositions can be used in the area of monitoring and characterizing injury and healing of damaged spinal cords. In a typical spinal cord injury, as occurs in an automobile accident for example, there may also be damage to tissue surrounding the spinal cord. It is thought that the process of healing of the surrounding tissue may be deleterious to healing of the spinal cord. It is thought that formation of neovasculature in the surrounding tissue, as occurs in healing of the surrounding tissue, may inhibit healing of the spinal cord. It is thought that by inhibiting healing of the surrounding tissue, and the formation of neovasculature in the surrounding tissue, the spinal cord may heal. Subsequently, the surrounding tissue may heal. The compositions of contrast-enhancing agents described here may be useful for monitoring the healing and inhibition of healing of the tissue surrounding the spinal cord.

There may be a variety of other applications for the compositions described herein. For example, the compositions may be used in detection and monitoring of inflammation, reperfusion injuries, and the like.

Additionally, the liposomes which comprise the compositions of contrast-enhancing agents can be targeted to desired cells and tissues in the body of a subject by, for example, attaching antibodies to the surface of the liposomes. This targeting may result in enhanced contrast to the targeted areas of the body.

The compositions of contrast-enhancing agents may have a relatively long residence time in the body, low extravasation, except in those areas of the vasculature that are leaky as described above, may be relatively nontoxic to the kidneys and may be used to target specific areas of the body. Additionally, the traditional osmolality related toxicity problems associated with ionic contrast-enhancing media generally are not an issue with the liposomal encapsulates since the high osmolality phase is interior to the liposomes and generally is not exposed to the blood.

### EXAMPLES

### Example 1

### Pilot Scale Preparation of PEGylated Liposomes Containing Iohexol Using a Pleated Filter

Example liposomal iohexol formulations can be prepared as follows. Briefly, a lipid mixture (150 mM) of 1,2-Dipalmitoly-sn-glycero-3-phosphocholine (DPPC), cholesterol (chol) and N-(carbonyl-methoxypolyethyleneglycol 2000)-1,2-distearoyl-sn-glycero-3-phosphoethanolamine (DSPE-MPEG2000), in a 55:40:5 molar ratio, was dissolved in ethanol at 70° C. The ethanol solution was hydrated with iohexol solution (350 mg I/mL) for 90 minutes. Liposomes were extruded through one or more 10 inch length GE polycarbonate track-etch pleated filters (that is, the liposomes were extruded through one such filter but, if that filter clogged or otherwise became unusable, the filter was replaced with a different filter for subsequent extrusions). The pleated filters were encased in a stainless steel (SS-316) housing. At least eight extrusion passes were performed through one or more 200 nm pore-size pleated filters at an average pressure of 20 psi and an average flow rate of 348 LPH/(m² of pore area). Subsequently, at least five extrusion passes were performed through one or more 100 nm pore-size pleated filters at an average pressure of 34 psi and an average flow rate of 348 LPH/(m² of pore area). Un-encapsulated iodine was removed using a MicroKros® diafiltration module of 500 kDa cut-off.

### Example 2

### Pilot Scale Preparation of PEGylated Liposomes Containing Iohexol Using a Flat-Stock Filter

Example liposomal iohexol formulations can be prepared as follows. Briefly, a lipid mixture (150 mM) of 1,2-Dipalmitoly-sn-glycero-3-phosphocholine (DPPC), cholesterol (chol) and N-(carbonyl-methoxypolyethyleneglycol 2000)-1,2-distearoyl-sn-glycero-3-phosphoethanolamine (DSPE-MPEG2000), in a 55:40:5 molar ratio, was dissolved in ethanol at 70° C. The ethanol solution was hydrated with iohexol solution (350 mg I/mL) for 90 minutes. The solution was subsequently extruded on a 800 mL Lipex Thermoline extruder with a minimum of eight passes through one or more 200 nm Nuclepore membranes (flat-stock filter) at an average pressure of 200 psi and an average flow rate of 6671 LPH/(m² of pore area). Subsequently, at least five extrusion passes were performed through one or more 100 nm Nuclepore membranes at an average pressure of 240 psi and an average flow rate of 17162 LPH/(m² of pore area). Un-encapsulated iodine was removed using a MicroKros® diafiltration module of 500 kDa cut-off.

### Example 3

### Lab Scale Preparation of PEGylated Liposomes Containing Iohexol Using a Flat-Stock Filter

Example liposomal iohexol formulations can be prepared as follows. Briefly, a lipid mixture (150 mM) of 1,2-Dipalmitoly-sn-glycero-3-phosphocholine (DPPC), cholesterol (chol) and N-(carbonyl-methoxypolyethyleneglycol 2000)-1,2-distearoyl-sn-glycero-3-phosphoethanolamine (DSPE-MPEG2000), in a 55:40:5 molar ratio, was dissolved in ethanol at 70° C. The ethanol solution was hydrated with iohexol solution (350 mg I/mL) for 90 minutes. The solution was subsequently extruded on a 10 mL Lipex Thermoline extruder with a minimum of eight passes through one or more 200 nm Nuclepore membranes at an average pressure of 200 psi and an average flow rate of 18676 LPH/(m² of pore area). Subsequently, at least five extrusion passes were performed through one or more 100 nm Nuclepore membranes at an average pressure of 200 psi or higher and an average flow rate of 35017 LPH/(m² of pore area). Un-encapsulated iodine was removed using a MicroKros® diafiltration module of 500 kDa cut-off.

### Example 4

### Lab Scale Preparation of PEGylated Liposomes Containing Iohexol Using a Flat-Stock Filter

Example liposomal iohexol formulations can be prepared as follows. Briefly, a lipid mixture (150 mM) of 1,2-Dipalmitoly-sn-glycero-3-phosphocholine (DPPC), cholesterol (chol) and N-(carbonyl-methoxypolyethyleneglycol 2000)-1,2-distearoyl-sn-glycero-3-phosphoethanolamine (DSPE-MPEG2000), in a 55:40:5 molar ratio, was dissolved in ethanol at 70° C. The ethanol solution was hydrated with iohexol solution (350 mg I/mL) for 90 minutes. The solution was subsequently extruded on a 10 mL Lipex Thermoline extruder with a minimum of eight passes through one or more 200 nm Nuclepore membranes at an average pressure of 20 psi and an average flow rate of 2594 LPH/(m² of pore area). Subsequently, at least five extrusion passes were performed through one or more 100 nm Nuclepore membranes at an average pressure of 45 psi or higher and an average flow rate of 973 LPH/(m² of pore area). Un-encapsulated iodine was removed using a MicroKros® diafiltration module of 500 kDa cut-off.

Table 1, below, summarizes the different filter sizes, flow rates, and extrusion pressures used to make the formulations. The flow rates are normalized by comparing the flow rate per total pore area of the filter (LPH/m²) and the flow rate per total filter area (LPH/m²).

| | Example 1 | | Example 2 | | Example 3 | | Example 4 | |
|---|---|---|---|---|---|---|---|---|
| Pore size | 200 | 100 | 200 | 100 | 200 | 100 | 200 | 100 |
| (nm) | | | | | | | | |
| Filter size | | | 90 | 90 | 25 | 25 | 25 | 25 |
| (mm) | | | | | | | | |
| Pore density | 3.00E+08 | 4.00E+08 | 3.00E+08 | 4.00E+08 | 3.00E+08 | 4.00E+08 | 3.00E+08 | 4.00E+08 |
| (pores/cm²) | | | | | | | | |
| Filter area | 16000.00 | 16000.00 | 63.62 | 63.62 | 4.91 | 4.91 | 4.91 | 4.91 |
| (cm²) | | | | | | | | |
| Total pore area | 1.51E+03 | 5.03E+02 | 6.00E+00 | 2.00E+00 | 4.63E−01 | 1.54E−01 | 4.63E−01 | 1.54E−01 |
| (cm²) | | | | | | | | |
| % of pore area | 9.42 | 3.14 | 9.42 | 3.14 | 9.42 | 3.14 | 9.42 | 3.14 |
| on filter | | | | | | | | |
| Flow rate | 52.5 | 17.5 | 4 | 3.43 | 0.864 | 0.54 | 0.12 | 0.015 |
| (LPH) | | | | | | | | |
| Flow rate per | 348 | 348 | 6671 | 17162 | 18676 | 35017 | 2594 | 973 |
| total pore area | | | | | | | | |
| (LPH/m²) | | | | | | | | |
| Flow rate per | 33 | 11 | 629 | 539 | 1760 | 1100 | 244 | 31 |
| total filter area | | | | | | | | |
| (LPH/m²) | | | | | | | | |
| Extrusion | 20 | 34 | 200 | 240 | 200 | 200 | 20 | 45 |
| Pressure | | | | | | | | |
| (psi) | | | | | | | | |
| Liposome | Leaky | Leaky | Non− | Non− | Non− | Non− | Leaky | Non− |
| stability (leaky | | | leaky | leaky | leaky | leaky | | leaky |
| vs. non-leaky) | | | | | | | | |

It should be noted that the use herein of the term “pilot scale” typically means reaction volumes in the range of about 100 mL to several kiloliters. The term “lab scale” typically means reaction volumes of less than about 100 mL.

The following formulations where prepared and tested as described in Examples 5 and 6 below:

Formulation A: A lipid mixture (150 mM) of 1,2-Dipalmitoly-sn-glycero-3-phosphocholine (DPPC), cholesterol (chol) and N-(carbonyl-methoxypolyethyleneglycol 2000)-1,2-distearoyl-sn-glycero-3-phosphoethanolamine (DSPE-MPEG2000), in a 55:40:5 molar ratio, was dissolved in ethanol at 70° C. The ethanol solution was hydrated with iohexol solution (350 mg I/mL) for 90 minutes. Liposomes were extruded through one or more 10 inch length GE polycarbonate track-etch pleated filters. The pleated filters were encased in a stainless steel (SS-316) housing. Sixteen passes were performed through one or more 200 nm pore-size pleated filters at an average pressure of 20 psi and an average flow rate of 348 LPH/(m² of pore area). Subsequently, 10 passes were performed through one or more 100 nm pore-size pleated filters at an average pressure of 34 psi and an average flow rate of 348 LPH/(m² of pore area). Un-encapsulated iodine was removed using a MiniKros® diafiltration module of 500 kDa cut-off.

Formulation B: A lipid mixture (150 mM) of 1,2-Dipalmitoly-sn-glycero-3-phosphocholine (DPPC), cholesterol (chol) and N-(carbonyl-methoxypolyethyleneglycol 2000)-1,2-distearoyl-sn-glycero-3-phosphoethanolamine (DSPE-MPEG2000), in a 55:40:5 molar ratio, was dissolved in ethanol at 70° C. The ethanol solution was hydrated with iohexol solution (350 mg I/mL) for 90 minutes. Liposomes were extruded through one or more 10 inch length GE polycarbonate track-etch pleated filters. The pleated filters were encased in a stainless steel (SS-316) housing. Sixteen passes were performed through one or more 200 nm pore-size pleated filters at an average pressure of 20 psi and an average flow rate of 348 LPH/(m² of pore area). Subsequently, 10 passes were performed through one or more 100 nm pore-size pleated filters at an average pressure of 34 psi and an average flow rate of 348 LPH/(m² of pore area). Finally, six passes were performed through a 100 nm Nuclepore membrane at an average pressure of 30 psi and an estimated flow rate of 1000 LPH/(m² of pore area). Un-encapsulated iodine was removed using a MicroKros® diafiltration module of 500 kDa cut-off.

Formulation C: A lipid mixture (150 mM) of 1,2-Dipalmitoly-sn-glycero-3-phosphocholine (DPPC), cholesterol (chol) and N-(carbonyl-methoxypolyethyleneglycol 2000)-1,2-distearoyl-sn-glycero-3-phosphoethanolamine (DSPE-MPEG2000), in a 55:40:5 molar ratio, was dissolved in ethanol at 70° C. The ethanol solution was hydrated with iohexol solution (350 mg I/mL) for 90 minutes. Liposomes were extruded through one or more 10 inch length GE polycarbonate track-etch pleated filters. The pleated filters were encased in a stainless steel (SS-316) housing. Sixteen passes were performed through one or more 200 nm pore-size pleated filters at an average pressure of 20 psi and an average flow rate of 348 LPH/(m² of pore area). Subsequently, 10 passes were performed through one or more 100 nm pore-size pleated filters at an average pressure of 34 psi and an average flow rate of 348 LPH/(m² of pore area). Finally, six passes were performed through a 100 nm Nuclepore membrane at an average pressure of at least 100 psi and an estimated flow rate of 17000 LPH/(M² of pore area). Un-encapsulated iodine was removed using a MicroKros® diafiltration module of 500 kDa cut-off.

Formulation D: A lipid mixture (150 mM) of 1,2-Dipalmitoly-sn-glycero-3-phosphocholine (DPPC), cholesterol (chol) and N-(carbonyl-methoxypolyethyleneglycol 2000)-1,2-distearoyl-sn-glycero-3-phosphoethanolamine (DSPE-MPEG2000), in a 55:40:5 molar ratio, was dissolved in ethanol at 70° C. The ethanol solution was hydrated with iohexol solution (350 mg I/mL) for 90 minutes. Liposomes were extruded through one or more 10 inch length GE polycarbonate track-etch pleated filters. The pleated filters were encased in a stainless steel (SS-316) housing. Seven passes were performed through one or more 200 nm pore-size pleated filters at an average pressure of 20 psi and an average flow rate of 348 LPH/(m² of pore area). Un-encapsulated iodine was removed using a MiniKros® diafiltration module of 500 kDa cut-off.

Formulation E: A lipid mixture (150 mM) of 1,2-Dipalmitoly-sn-glycero-3-phosphocholine (DPPC), cholesterol (chol) and N-(carbonyl-methoxypolyethyleneglycol 2000)-1,2-distearoyl-sn-glycero-3-phosphoethanolamine (DSPE-MPEG2000), in a 55:40:5 molar ratio, was dissolved in ethanol at 70° C. The ethanol solution was hydrated with iohexol solution (350 mg I/mL) for 90 minutes. The solution was subsequently extruded on a 10 mL Lipex Thermoline extruder with seven passes through one or more 200 nm Nuclepore membranes at an average pressure of 20 psi or higher and an average flow rate of 2594 LPH/(m² of pore area). Un-encapsulated iodine was removed using a MicroKros® diafiltration module of 500 kDa cut-off.

Formulation F: A lipid mixture (150 mM) of 1,2-Dipalmitoly-sn-glycero-3-phosphocholine (DPPC), cholesterol (chol) and N-(carbonyl-methoxypolyethyleneglycol 2000)-1,2-distearoyl-sn-glycero-3-phosphoethanolamine (DSPE-MPEG2000), in a 55:40:5 molar ratio, was dissolved in ethanol at 70° C. The ethanol solution was hydrated with iohexol solution (350 mg I/mL) for 90 minutes. The solution was subsequently extruded on a 10 mL Lipex Thermoline extruder with seven passes through one or more 200 nm Nuclepore membranes at an average pressure of 100 psi or higher and an estimated flow rate of 9000 LPH/(m² of pore area). Un-encapsulated iodine was removed using a MiniKros® diafiltration module of 500 kDa cut-off.

Formulation G: A lipid mixture (150 mM) of 1,2-Dipalmitoly-sn-glycero-3-phosphocholine (DPPC), cholesterol (chol) and N-(carbonyl-methoxypolyethyleneglycol 2000)-1,2-distearoyl-sn-glycero-3-phosphoethanolamine (DSPE-MPEG2000), in a 55:40:5 molar ratio, was dissolved in ethanol at 70° C. The ethanol solution was hydrated with iohexol solution (350 mg I/mL) for 90 minutes. The solution was subsequently extruded on a 10 mL Lipex Thermoline extruder with eight passes through one or more 200 nm Nuclepore membranes at an average pressure of 100 psi or higher and an estimated flow rate of 9000 LPH/(m² of pore area). Subsequently, at five extrusion passes were performed through one or more 100 nm Nuclepore membranes at an average pressure of 100 psi or higher and an average flow rate of 17000 LPH/(m² of pore area). Un-encapsulated iodine was removed using a MicroKros® diafiltration module of 500 kDa cut-off.

Formulation H: A lipid mixture (150 mM) of 1,2-Dipalmitoly-sn-glycero-3-phosphocholine (DPPC), cholesterol (chol) and N-(carbonyl-methoxypolyethyleneglycol 2000)-1,2-distearoyl-sn-glycero-3-phosphoethanolamine (DSPE-MPEG2000), in a 55:40:5 molar ratio, was dissolved in ethanol at 70° C. The ethanol solution was hydrated with iohexol solution (350 mg I/mL) for 90 minutes. The solution was subsequently extruded on a 800 mL Lipex Thermoline extruder with eight passes through one or more 200 nm Nuclepore membranes at an average pressure of 200 psi or higher and an average flow rate of 6671 LPH/(m² of pore area). Subsequently, five extrusion passes were performed through one or more 100 nm Nuclepore membranes at an average pressure of 240 psi or higher and an average flow rate of 17162 LPH/(m² of pore area). Un-encapsulated iodine was removed using a MicroKros® diafiltration module of 500 kDa cut-off

Table 2 compares the properties of the liposomes formed in Formulations A and

H, each prepared on a pilot scale:

| | Formulation A | Formulation H |
|---|---|---|
| Final Lipid (mM) | 120.63 | 186 |
| Final Iodine (mg/mL) | 95.6 | 95 |
| Average Size (nm) | 137 | 101 |

### Example 5

### In Vitro Stability of PEGylated Liposomes Containing Iohexol

The in vitro stability of liposomal iodinated contrast enhancing agent formulations can be determined by measuring the leakage of iohexol from liposomal iohexol formulations in saline solution and bovine plasma. For saline testing, the liposomal iohexol formulations were diluted 50-fold in saline solution and divided into two aliquots. One aliquot was incubated at room temperature for 120 minutes. The other aliquot was incubated at 37° C. for 120 minutes. Subsequently, both the samples were dialyzed using centricon tubes (10,000 MWCO). The filtrate was assayed for iodine content to determine the amount of iodine leak.

To measure stability in plasma, 0.1 mL of the liposomal iohexol formulation was mixed with 1 mL of bovine plasma and incubated at 37° C. for 120 minutes. The mixture was then dialyzed against 300 mOsm saline for 20 hours. The external saline phase was assayed for iodine leakage.

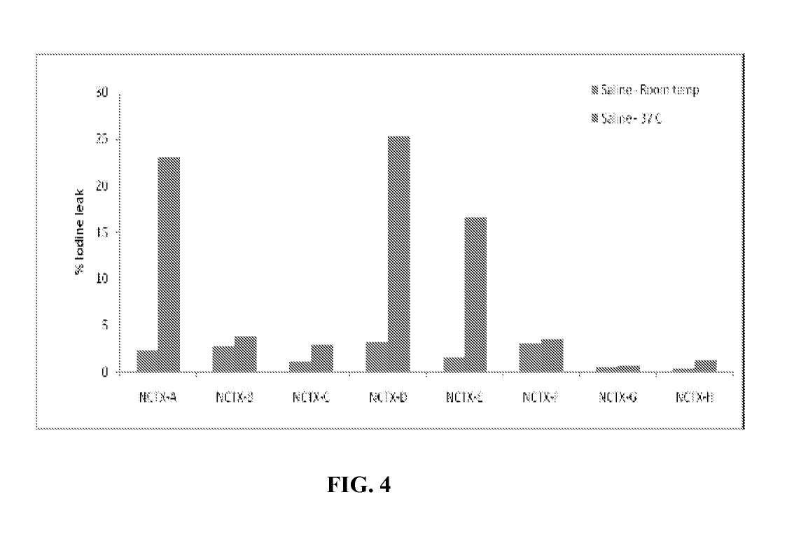

FIG. 4 details the amount of iodine leakage found in formulations A-H, prepared as described above. All of the formulations demonstrated low iodine leak in saline at room temperature. However, formulations extruded using the pleated filters at low pressure and low flow rate (e.g., 34 psi/348 LPH/(m² of pore area) and 20 psi/348 LPH/(m² of pore area) for Formulations A and D, respectively) demonstrated high iodine leak at 37° C., indicating that a less stable liposome was formed. Interestingly, pilot scale formulations that were subsequently extruded on the lab scale at low pressure/high flow rate and high pressure/high flow rate (e.g., 30 psi/1000 LPH/(m² of pore area) and 100 psi/17000 LPH/(m² of pore area) for Formulations B and C, respectively) demonstrated low iodine leak at 37° C. Formulations that were prepared using the flat-stock filters at high pressure/high flow rate, both lab scale and pilot scale, demonstrated the minimum iodine leak in both saline conditions (e.g, 100 psi/9000 LPH/(m² of pore area) and 100 psi/17000 LPH/(m² of pore area) for Formulation G and 200 psi/6671 LPH/(m² of pore area) and 240 psi/17162 LPH/(m² of pore area) for Formulation H.

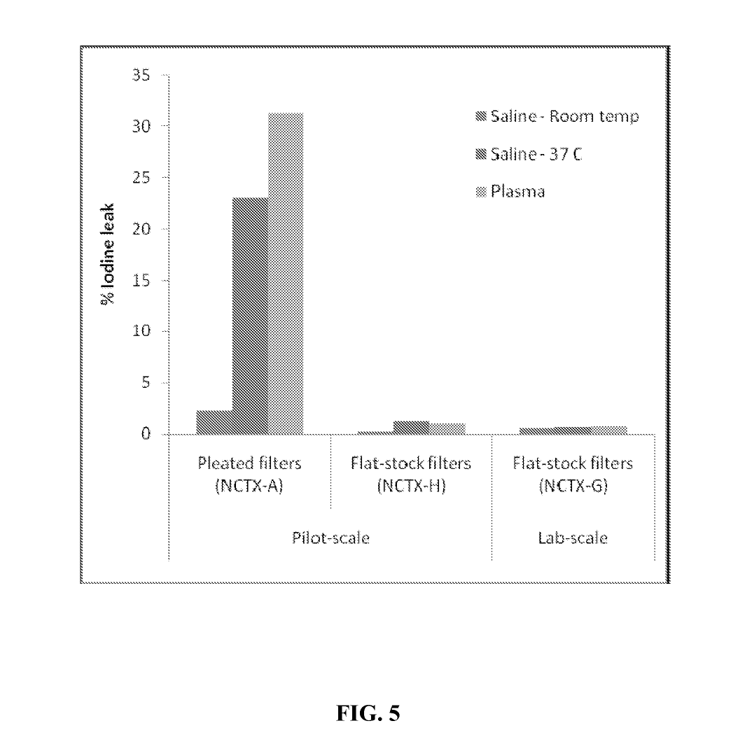

FIG. 5 compares the stability of the formulations prepared by two different extrusion methods. The formulations prepared on a pilot scale using pleated filters at low pressure and a low flow rate demonstrated high iodine leak in saline at 37° C., both in saline and in plasma (Formulation A). By contrast, the formulation prepared on a pilot scale at high pressure and with a high flow rate using the flat stock filters demonstrated a very low leak under all conditions (Formulation H). The formulation prepared on a lab scale at high pressure and with a high flow rate using the flat stock filters also demonstrated a very low leak under all conditions (Formulation G).

FIG. 5 also compares the stability for formulations prepared on two different scales. The in vitro performances of formulations prepared using flat stock filters were very similar at the lab scale and the pilot scale (formulations G and H). Both of the formulations demonstrated low iodine leak under all testing conditions.

### Example 6

### In Vivo Studies Using Imaging of PEGylated Liposomes Containing Iohexol in a Mouse

In vivo performance of the liposomal formulations was tested in C57BL6/J mice. 0.5 mL of the formulations was slowly injected intravenously via the tail vein. Micro-CT imaging of the mouse was done at 30, 60, and 120 minutes post-injection. Signal was measured in the descending aorta, liver, spleen, and bladder.

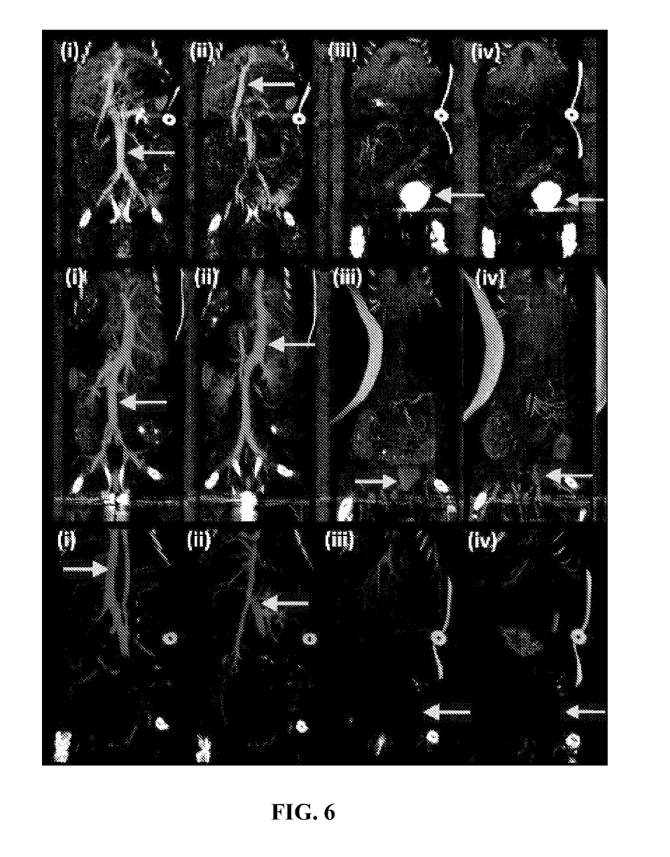

FIG. 6 is a composite of coronal maximum intensity projection images showing the relative stability performances for in vivo testing. Similar to the in vitro plasma leak, the pilot scale formulation prepared at low pressure and low flow rate (Formulation A) using pleated filters showed significant leak in vivo (FIG. 6, top row). While the abdominal vascular was enhanced (i and ii), significant bladder signal was observed (iii and iv). Conversely, formulations prepared using the flat stock filters at high pressure and high flow rates (Formulations G (bottom row) and H (middle row)) showed enhancement of only the vasculature (i and ii) with negligible bladder enhancement (iii and iv).

The descriptions given above are given as examples and are not meant to be limiting in nature. One of skill in the art will appreciate that certain modifications and alterations may be used upon reading and understanding the preceding description. It is intended that the embodiments described herein be construed as including all such alterations and modifications insofar as they come within the scope of the appended claims or the equivalence thereof.

1. A method for making a composition, comprising:
selecting one or more solutions containing one or more nonradioactive contrast-enhancing agents;
forming liposomes in the presence of the one or more solutions containing one or more nonradioactive contrast-enhancing agents to provide a solution of liposomes that at least partially encapsulate the one or more nonradioactive contrast-enhancing agents; and
passing the solution of liposomes that at least partially encapsulate the one or more nonradioactive contrast-enhancing agents through a filter at at least one of a high flow rate and a high pressure.

2. The method of claim 1, the passing comprising passing through the filter at a pressure of at least 45 psi.

3. The method of claim 1, the passing comprising passing through the filter at a pressure of at least about 100 psi.

4. The method of claim 1, the passing comprising passing through the filter at a pressure of at least about 200 psi.

5. The method of claim 1, the passing comprising passing through the filter at a flow rate per total pore area of at least about 900 LPH/m² and a pressure of at least 40 psi.

6. The method of claim 1, the passing comprising passing through the filter at a flow rate per total pore area of at least about 900 LPH/m².

7. The method of claim 1, the passing comprising passing through the filter at a flow rate per total pore area of at least about 5000 LPH/m².

8. The method of claim 1, the passing comprising passing through the filter at a flow rate per total pore area of at least about 15000LPH/m².

9. The method of claim 1, wherein the filter has from about 100 nm to about 200 nm pore diameter.

10. The method of claim 1, the passing comprising passing through the filter at a pressure of greater than or equal to about 100 psi from about 5 to about 30 times.

11. The method of claim 1, wherein the liposomes that at least partially encapsulate the one or more nonradioactive contrast-enhancing agents exhibit less than about a 5% iodine leak in saline at 37° C. after being passed through the filter.

12. The method of claim 1, wherein the liposomes that at least partially encapsulate the one or more nonradioactive contrast-enhancing agents encapsulate from about 37 to about 200 milligrams of iodine per milliliter of a suspension medium in which the liposomes that at least partially encapsulate the one or more nonradioactive contrast-enhancing agents are suspended.

13. The method of claim 1, wherein the liposomes that at least partially encapsulate the one or more nonradioactive contrast-enhancing agents encapsulate from about 80 to about 110 milligrams of iodine per milliliter of a suspension medium in which the liposomes that at least partially encapsulate the one or more nonradioactive contrast-enhancing agents are suspended.

14. The method of claim 1, wherein the liposomes that at least partially encapsulate the one or more nonradioactive contrast-enhancing agents encapsulate from about 90 to about 100 milligrams of iodine per milliliter of a suspension medium in which the liposomes that at least partially encapsulate the one or more nonradioactive contrast-enhancing agents are suspended.

15. The method of claim 1, wherein the liposomes that at least partially encapsulate the one or more nonradioactive contrast-enhancing agents have an average diameter of less than 150 nm.

16. The method of claim 1, wherein the method further comprises concentrating the solution of liposomes that at least partially encapsulate the one or more nonradioactive contrast-enhancing agents by at least about 10%.

17. The method of claim 1, the forming comprising at least one of: hydrating dried lipids in the presence of the one or more solutions containing one or more nonradioactive contrast-enhancing agents; mixing a volatile organic solution of lipids with the one or more solutions containing one or more nonradioactive contrast-enhancing agents; and dialyzing an aqueous solution of one or more of lipids, detergents, and surfactants in the presence of the one or more solutions containing one or more nonradioactive contrast-enhancing agents to remove the one or more lipids, detergents, and surfactants.

18. The method of claim 1, wherein the liposomes are comprised of cholesterol, at least one lipid or phospholipid, and at least one phospholipid which is derivatized with a polymer chain.

19. The composition made according to the method of claim 1.

20. A method for making a composition, comprising:
selecting a solution containing an iodinated contrast-enhancing agent;
forming liposomes in the presence of the iodinated contrast-enhancing agent to provide a solution of liposomes that is associated with the iodinated contrast-enhancing agents; and
passing the solution of liposomes that is associated with the iodinated contrast-enhancing agents through a filter at a flow rate per total pore area of from about 900 LPH/m² to about 50,000 LPH/m²,
wherein the liposomes have up to about a 5% iodine leak in saline at 37° C. after being passed through the filter.

21. The method of claim 20, wherein the passing further comprises passing through the filter at a pressure of at least about 100 psi.

22. The method of claim 20, wherein the filter has from about 100 nm to about 200 nm pore diameter.

23. The method of claim 20, wherein the liposomes are comprised of at least one lipid or phospholipid, cholesterol, and at least one phospholipid which is derivatized with a polymer chain.

24. The method of claim 23, wherein the at least one lipid or phospholipid, the cholesterol, and the at least one phospholipid which is derivatized with a polymer chain are present in a molar ratio of about 55:40:5.

25. The method of claim 23, wherein the at least one lipid or phospholipid is present in an amount of about 55 to about 75 mol %, the cholesterol is present in an amount of about 25 to 40 mol %, and the at least one phospholipid which is derivatized with a polymer chain is present in an amount of about 1 to 20 mol %.

26. A composition made according to the method of claim 20.

27. A method of making a liposomal composition, comprising:
forming liposomes in the presence of iodinated contrast enhancing agents to form liposomes which at least partially encapsulate the iodinated contrast enhancing agents, the liposomes comprising at least one lipid or phospholipid present in an amount of about 55 to about 75 mol %, cholesterol present in an amount of about 25 to 40 mol %, and at least one phospholipid which is derivatized with a polymer chain present in an amount of about 1 to 20 mol %; and
extruding the liposomes which at least partially encapsulate the iodinated contrast enhancing agents through a filter at a high flow rate and at a high pressure,
wherein the extruded liposomes which at least partially encapsulate the iodinated contrast enhancing agents have an average diameter of less than 150 nm and a concentration of at least 80 milligrams of iodine per milliliter of a suspension medium in which the liposomes are suspended.

28. The method of claim 27, wherein the extruding comprises extruding through the filter at a pressure of at least 100 psi.

29. The method of claim 27, wherein the extruding comprises extruding through the filter at a flow rate per total pore area of at least about 900 LPH/m².

30. A liposomal composition prepared according to the method of claim 27.
